(19) United States
(12) Patent Application Publication
(10) Pub. No.: US 2026/0174668 A1
(43) Pub. Date: Jun. 25, 2026

(54) IMPROVED INDIGO DYEING METHODS

(71) Applicant: HENKEL AG & CO. KGAA, Düsseldorf (DE)

(72) Inventors: Skrollan KLAAS, Hamburg (DE); Jana Marie ROESEKE, Tornesch (DE); Nele DALLMANN, Hamburg (DE); Fabian STRASKE, Hamburg (DE); Thomas HIPPE, Appen (DE)

(21) Appl. No.: 19/128,929

(22) PCT Filed: Oct. 24, 2023
(86) PCT No.: PCT/EP2023/079535

§ 371 Date: May 9, 2025

(30) Foreign Application Priority Data
Nov. 11, 2022 (DE) 10 2022 211 975.4

(51) Int. Cl. A61K 8/97; A61K 8/46; A61K 8/66; A61Q 5/10

(52) U.S. Cl. CPC A61K 8/97; A61K 8/46; A61K 8/66; A61Q 5/10; A61K 2800/591; A61K 2800/884

(57) ABSTRACT

Methods for dyeing keratinous fibers, in particular human hair, using an indigo-producing plant powder and a post-treatment agent are described.

The invention relates to methods for dyeing keratinous fibers, in particular human hair, using an indigo-producing plant powder and a post-treatment agent.

The desire to change one's hair color is a great need for many consumers. To satisfy this need, the cosmetic industry provides a diverse range of products. Hair coloring agents that achieve particularly long-lasting coloring with high coverage are usually oxidation dyes. These use oxidizing agents, which can damage the hair structure. Certain cationic direct azo dyes are also capable of producing hair color changes with excellent fastness properties. However, the azo dyes mentioned are synthetic dyes.

A growing number of consumers are demanding hair dyes and hair dyeing methods based on natural dyes, even though these agents and methods are often inferior to the aforementioned agents and methods in terms of fastness properties, coverage, and color variety.

In addition to dyeing with henna, obtained from the plant Lawsonia inermis, the dyeing of hair with plants from which indigo can be produced has also been known for a long time.

### PRIOR ART

There has been no lack of efforts in the prior art to improve the dyeing of keratin fibers, in particular human hair, with indigo.

The addition of natural dyes to oxidative hair dyes is also popular. The dyeing cream is mixed with an aqueous preparation containing hydrogen peroxide shortly before application to the hair. The dyeing cream predominantly contains oxidation dye precursors, for example toluene-2,5-diamine sulfate, 4-chlororesorcinol, 2-methylresorcinol, 2-amino-4-hydroxyethylaminoanisole sulfate, 4-amino-2-hydroxytoluene, 4-amino-m-cresol and m-aminophenol; the content of natural dyes only serves to adjust the shade. The coloration thus achieved is essentially a conventional oxidative coloration with high fastness properties. The problem of the lack of adhesion of the natural dye is of minor importance in such products.

When dyeing with indigo-producing plants, it is often observed that the coloration changes continuously over a period of up to 2 weeks after application of the dye and that the final color tone only remains after this period. Starting from a concentration of 2 wt. % of indigo-producing plant powder in water, the color tone on the hair changes in the first few days after dyeing from an initial turquoise-blue tone to a redder and darker tone, creating a purple color impression.

WO 2015/082482 A1 discloses a method for dyeing hair with powdered plant parts from plants that produce indigo, wherein the hair is post-treated with a strongly alkaline composition after the indigo dyeing, which can be carried out in the presence of hydrogen peroxide. This is intended to accelerate the development of the final color tone and to prevent any change in color after the actual dyeing process has been completed. However, the alkaline pH and the oxidizing agent damage the hair.

### OBJECT

One object of the present invention was to provide methods for dyeing keratinous fibers, in particular human hair, using plants that produce indigo, by means of which the final color result of the indigo coloration is obtained as quickly as possible after completion of the actual dyeing process.

Surprisingly, it was found that with the dyeing methods described in the claims, using plants that produce indigo, colorations can be achieved with which the final color result of the indigo dyeing is obtained as quickly as possible after completion of the actual dyeing process.

A first object of the present invention is therefore a method for the non-oxidative dyeing of keratin fibers, in particular human hair, comprising the following method steps in the specified sequence:
a) providing an aqueous dye composition (A) which contains at least one indigo-producing plant powder i), is free of cysteine and has a pH of 2.0 to 10.0, preferably 4.0 to 9.0, particularly preferably 5.0 to 8.0, extremely preferably 6.0 to 7.4, in each case measured at 20° C.,
b) applying the aqueous dye composition (A) to the keratin fibers,
c) allowing to act for a time of from 30 seconds to 60 minutes, preferably 5 to 45 minutes, particularly preferably 15 to 30 minutes,
d) rinsing the keratin fibers with water,
e) optionally drying the keratin fibers,
f) immediately thereafter, within a time of zero seconds to 30 minutes, preferably 10 seconds to 10 minutes, particularly preferably 1 to 5 minutes, treating the keratin fibers with one of the following post-treatment steps:
   f)i. rinsing or soaking the keratin fibers with additive-free water for a period of 5 to 120 minutes,
   f)ii. rinsing or soaking the keratin fibers with an aqueous cysteine solution (C) having a pH of less than 7.5 measured at 20° C. for a period of 5 to 120 minutes,
   f)iii. rinsing or soaking the keratin fibers with an aqueous composition (G) containing at least one glycosylase (EC 3.2) for a period of 5 to 120 minutes,
   f)iv. rinsing or soaking the keratin fibers with an aqueous composition (L) containing laccase (EC 1.10.3.2) for a period of 5 to 120 minutes,
   f)v. rinsing or soaking the keratin fibers with an aqueous composition (AT) containing, relative to the weight thereof, 1-20 wt. % of an anionic surfactant and having a pH in the range of 3-6, measured at 20° C., for a period of 5 to 120 minutes,
wherein no hydrogen peroxide and no ions and compounds of metals, other than alkali metals and alkaline-earth metals, are used in the method.

The terms “dye used according to the invention” and dye composition (A) are used synonymously in the present application.

Dyes preferably used according to the invention are characterized in that the indigo-producing plant(s) is/are selected from at least one species of the following genera:
Indigofera, in particular Indigofera tinctoria, Indigo suffruticosa, Indigofera articulata, Indigofera arrecta, Indigofera heterantha “gerardiana”, Indigofera argentea or Indigofera longiracemosa;
Isatis, in particular Isatis tinctoria (woad);
Persicaria, in particular Persicaria tinctoria (dyer's knotweed);
Wrightia, in particular Wrightia tinctoria;
Calanthe, in particular Calanthe veratrifolia; and
Baphicacanthus cusia, synonym Strobilanthes cusia.

Particularly preferred plants that produce indigo are selected from the genus Indigofera and in particular from Indigofera tinctoria.

The plants of the abovementioned genera and species contain no indigo or indirubin, or only small amounts thereof. Instead, the plants contain the compound indican, a precursor of indigo and indirubin. The parts of the abovementioned plant genera and species that are rich in indican and also contain indigo are the leaves of these plants. For use as a dye for keratin fibers, the plant leaves of the abovementioned plant genera and species are dried and ground and used as plant powder.

Plant powders preferred according to the invention have a particle size of less than 500 μm, particularly preferably in the range of 120 μm-200 μm, extremely preferably in the range of 150 μm-180 μm. Other plant powders preferred according to the invention have a bulk density in the range of 0.20-0.60 g/cm³, particularly preferably in the range of 0.25-0.40 g/cm³. Further plant powders preferred according to the invention have, relative to the weight thereof, a moisture content of less than 10 wt. %, preferably of 0.1 to 6.0 wt. %, particularly preferably of 0.2 to 3.0 wt. %. The moisture content is determined after 3 hours of drying at 105° C.

It is preferred according to the invention that the powder of the indigo-producing plant, relative to the weight thereof, consists of at least 50 wt. %, preferably at least 80 wt. %, particularly preferably more than 80-100 wt. %, of the leaves of the plant. Further dyes preferably used according to the invention are characterized in that the powder of the indigo-producing plant has a particle size of less than 500 μm, particularly preferably in the range of 120 μm-200 μm, extremely preferably in the range of 150 μm-180 μm, and also a bulk density in the range of 0.20-0.60 g/cm³, particularly preferably in the range of 0.25-0.40 g/cm³ and, relative to the weight thereof, has a moisture content of less than 10 wt. %, preferably of 0.1 to 6.0 wt. %, particularly preferably of 0.2 to 3.0 wt. %.

Further dyes preferably used according to the invention are characterized in that the powder of the indigo-producing plant contains the compound indican in an amount of 2-5 wt. %, preferably 2.5-4.5 wt. %, particularly preferably 3-4.1 wt. %, relative to the weight of the powder.

Further dyes preferably used according to the invention are characterized in that at least one indigo-producing plant powder i) is present in a total amount of 1-80 wt. %, preferably 2-50 wt. %, particularly preferably 3-25 wt. %, extremely preferably 4-15 wt. %, exceptionally preferably 5-10 wt. %, exceptionally preferably 6-7 wt. %, relative to the weight of the agent.

Further dyes preferably used according to the invention are characterized in that powdered leaves of Indigofera tinctoria are present in an amount of 1-80 wt. %, preferably 2-50 wt. %, particularly preferably 3-25 wt. %, extremely preferably 4-15 wt. %, exceptionally preferably 5-10 wt. %, exceptionally preferably 6-7 wt. %, relative to the weight of the agent.

It has been found that the simultaneous application of an aqueous dispersion of at least one plant powder i), composed of at least one indigo-producing plant with cysteine, to keratin fibers leads to a red shift in the resulting coloration of the keratin fibers, which is undesirable in the present case.

The dye compositions (A) used according to the invention are therefore characterized in that they are free of cysteine, i.e. they do not contain cysteine.

Powders from the leaves of Indigofera tinctoria which are preferably used according to the invention react approximately neutrally in aqueous dispersion, with slight deviations between a slightly acidic and a slightly alkaline pH range. A 5 wt. % dispersion of ground leaves of Indigofera tinctoria in deionized water has a pH of 6.6 to 7.4, preferably 6.8 to 7.2, particularly preferably 6.9 to 7.1, in each case measured at 20° C.

The dyes used according to the invention also contain water. The water serves to disperse the plant powder and to dissolve the indican contained therein and make it available for the reaction to form indigo.

Dyes used according to the invention are characterized in that water is present in an amount of 19.9-95.0 wt. %, preferably 30.0-90.0 wt. %, particularly preferably 50.0-88.0 wt. %, extremely preferably 60.0-85.0 wt. %, relative to the weight of the agent.

In method step b), an aqueous dye composition (A) having a pH in the range of 2.0 to 10.0, preferably 4.0 to 9.0, particularly preferably 5.0 to 8.0, extremely preferably 6.0 to 7.4, in each case measured at 20° C., is applied to the keratin fibers to be dyed, which are preferably dry.

The dyes used according to the invention have a pH of 2.0 to 10.0, preferably 4.0 to 9.0, particularly preferably 5.0 to 8.0, extremely preferably 6.0 to 7.4, in each case measured at 20° C.

In a first preferred embodiment of the dyeing method according to the invention, the pH of the aqueous dye composition (A) is not influenced by the addition of an acid or a base, but rather corresponds to the pH generated by the dispersed plant powder. In a further preferred embodiment of the dyeing method according to the invention, the desired pH of the aqueous dye composition (A) is adjusted with the aid of an acid or a base. Preferred acids are selected from citric acid, lactic acid, gluconic acid, acetic acid, propionic acid, oxalic acid, malonic acid, succinic acid, glutaric acid, galactaric acid (mucic acid), tartaric acid, malic acid, sulfuric acid and phosphoric acid as well as mixtures of these acids. Preferred bases are sodium hydroxide, potassium hydroxide, arginine, lysine, monoethanolamine, triethanolamine, 2-amino-2-methylpropan-1-ol and mixtures of these bases.

In a further preferred embodiment of the dyeing method according to the invention, the desired pH of the aqueous dye composition (A) is adjusted with the aid of a buffer system selected from a mixture of a mid-strength or weak acid with its conjugated or corresponding base (or the respective salt) and a mixture of a mid-strength or weak base with its conjugated or corresponding acid.

Suitable corresponding acid-base pairs preferred according to the invention are those which stabilize the aqueous dye composition (A) used according to the invention in the pH range of 2.0 to 10.0, preferably 4.0 to 9.0, particularly preferably 5.0 to 8.0, extremely preferably 6.0 to 7.4, in each case measured at 20° C.

Buffer systems particularly preferred according to the invention for the aqueous dye composition (A) used according to the invention are selected from
ammonia/ammonium salt mixtures, wherein the ammonium salt is preferably selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate. Ammonium lactate, and mixtures of these ammonium salts, particularly preferably selected from ammonium chloride, ammonium hydrogen sulfate, ammonium sulfate, ammonium dihydrogen phosphate, diammonium hydrogen phosphate, ammonium phosphate, ammonium bicarbonate and ammonium carbonate, extremely preferably selected from ammonium chloride,
mixtures of hydrogen phosphate and dihydrogen phosphate, in particular the alkali metal salts of hydrogen phosphate and dihydrogen phosphate, particularly preferably the sodium salts and/or the potassium salts of hydrogen phosphate and dihydrogen phosphate,
mixtures of alkali metal hydrogen carbonate with alkali metal carbonate, in particular mixtures of sodium or potassium hydrogen carbonate with sodium or potassium carbonate,
mixtures of citric acid and the salts thereof, in particular the alkali metal citrates, in particular the sodium salts, in particular trisodium citrate,
mixtures of tartaric acid and the salts thereof, in particular alkali metal tartrates, in particular potassium salts, in particular potassium hydrogen tartrate,
mixtures of phthalic acid and the salts thereof, in particular potassium salts, in particular potassium hydrogen phthalate,
mixtures of lactic acid and the salts thereof, in particular lactic acid/sodium lactate mixtures,
mixtures of gluconic acid and the salts thereof, in particular gluconic acid/sodium gluconate mixtures,
mixtures of succinic acid and the salts thereof, in particular sodium salts, in particular sodium hydrogen succinate and disodium succinate, and
mixtures of malic acid and the salts thereof, in particular sodium salts, in particular sodium hydrogen malate and disodium malate, and
ammonia/ammonium salt mixtures, wherein the ammonium salt is preferably selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate. Ammonium lactate, and mixtures of these ammonium salts, particularly preferably selected from ammonium chloride, ammonium hydrogen sulfate, ammonium sulfate, ammonium dihydrogen phosphate, diammonium hydrogen phosphate, ammonium phosphate, ammonium bicarbonate and ammonium carbonate, extremely preferably selected from ammonium chloride.

Other buffer systems, e.g. acetic acid/sodium acetate, are in principle also suitable according to the invention. However, due to the vinegary odor, such a buffer is not acceptable for the production of a product for the cosmetics market.

For varying the pH, therefore, further dyes and dyeing methods preferably used according to the invention are characterized in that the aqueous dye composition (A) contains a buffer system for pH adjustment, selected from a mixture of a mid-strength or weak acid or base with its conjugated or corresponding base or corresponding acid.

Further dyeing methods preferred according to the invention are characterized in that the aqueous dye composition (A) contains a buffer system for pH adjustment in the basic range, selected from an ammonia/ammonium salt mixture. Preferred ammonium salts which buffer the strongly basic pH of the aqueous ammonia solution to a less basic pH are selected from ammonium chloride, ammonium bromide, ammonium hydrogen sulfate, ammonium sulfate, ammonium dihydrogen phosphate, diammonium hydrogen phosphate, ammonium phosphate, ammonium bicarbonate, ammonium carbonate, ammonium nitrate, ammonium acetate, ammonium glycolate, ammonium gluconate, ammonium tartrate. Ammonium lactate, and mixtures of these ammonium salts. Ammonium chloride, ammonium hydrogen sulfate, ammonium sulfate, ammonium dihydrogen phosphate, diammonium hydrogen phosphate, ammonium phosphate, ammonium bicarbonate and ammonium carbonate are particularly preferred. Ammonium chloride is extremely preferred.

In principle, however, other buffer systems are also suitable.

Further dyeing methods preferred according to the invention are characterized in that the aqueous dye composition (A) contains a buffer system for pH adjustment selected from a hydrogen phosphate salt/dihydrogen phosphate salt mixture. Suitable salts are the sodium salts and the potassium salts of hydrogen phosphate and dihydrogen phosphate. Hydrogen phosphate salt/dihydrogen phosphate salt mixtures make it possible to adjust pHs in the range of 7.1 to approximately 8.2.

For higher pHs, mixtures of sodium or potassium hydrogen carbonate with sodium or potassium carbonate are suitable. Further dyeing methods preferred according to the invention are therefore characterized in that the aqueous dye composition (A) contains a buffer system for pH adjustment, selected from a mixture of sodium or potassium hydrogen carbonate with sodium or potassium carbonate. Suitable salts are the sodium salts and the potassium salts of hydrogen carbonate and carbonate. Hydrogen carbonate salt/carbonate salt mixtures make it possible to adjust pHs in the range of approximately 9.0 to approximately 11.0.

Those skilled in the art can find the appropriate weights of buffer salts to adjust the desired pH in the relevant manuals.

Preferred dyes and dyeing methods according to the invention are further characterized in that no hydrogen peroxide is used in them.

Preferred dyes and dyeing methods according to the invention are further characterized in that no ions or compounds of metals, other than alkali metals and alkaline-earth metals, are used in them. The content of metal ions other than alkali metal ions and alkaline-earth metal ions which are present in trace amounts in the water used, e.g. in tap water or municipal water, is not taken into account here. Tap water can contain an average of 2 mg of copper ions per liter, i.e. approximately 0.0002 wt. % of copper ions. The maximum concentration of metal ions other than alkali metal ions and alkaline-earth metal ions in dyes preferred according to the invention is 0.00001-0.002 wt. %, preferably 0.0001 to 0.001 wt. %, particularly preferably at most 0.0007 wt. %, in each case relative to the dye.

If salts are to be present, the salts of alkali metals and alkaline-earth metals, in particular the salts of sodium, potassium and magnesium, preferably the salts of sodium and potassium, are suitable according to the invention.

According to the invention, the salts and compounds of aluminum, the transition metals and the lanthanides are particularly undesirable. The elemental form of the metals mentioned are also not used in the dyes and dyeing methods according to the invention.

In conventional dyes and dyeing methods with natural dyes, compounds, in particular salts, of metals other than alkali metals and alkaline-earth metals are often used to improve the adhesion of the natural dye to the keratin fibers. The present dyes and dyeing methods according to the invention can dispense with the use of ions and compounds of metals other than alkali metals and alkaline-earth metals.

Surprisingly, it was found that the development over time of the dyeing result on the keratin fibers can be accelerated if the dye composition (A) is allowed to act with a supply of heat, for example by means of a heat lamp or a drying hood or a hair dryer. Dyeing methods preferred according to the invention are therefore characterized in that the dye composition (A) is allowed to act with a supply of heat. Dyeing methods particularly preferred according to the invention are characterized in that the dye composition (A) is allowed to act with a supply of heat at a temperature of 25-60° C., particularly preferably at a temperature of 30-50° C., extremely preferably at a temperature of 35-40° C.

With regard to further preferred embodiments of the dyeing method according to the invention, what has been stated in relation to the dyes and dye compositions (A) used according to the invention applies, mutatis mutandis.

In order to preserve the hair-protecting potential of natural dyes, the claimed method is preferably limited to those methods in which the keratin fibers have not been treated with an oxidizing agent in a period of up to 7 days prior to the application of the dye composition (A) according to the invention.

The oxidizing agents which are conventionally used in hair cosmetics but which, according to the invention, are not intended to be used in hair treatment, not even as a pre-treatment, include hydrogen peroxide, persulfates, perbromates, percarbonates, perborates and percarbamides. The oxygen present in the ambient air does not constitute an oxidizing agent in the context of the invention.

In order to preserve the hair-protecting potential of natural dyes, the methods preferred according to the invention are preferably limited to those methods in which the keratin fibers have not been treated with a keratin-reducing compound in a period of up to 7 days prior to the application of the dye composition (A) according to the invention.

Preferably, the keratin fibers are dried after rinsing out the dye composition (A). Drying can take place without the active supply of heat. However, drying can also be carried out with a supply of heat at a temperature of 25-120° C., particularly preferably at a temperature of 30-80° C., extremely preferably at a temperature of 35-60° C. Heat is preferably supplied by a heat lamp, a drying rod, a drying hood, a straightening iron, or a hair dryer.

A further feature of the dyeing method according to the invention is that the dye composition (A) is allowed to act on the keratin fibers after application thereto for a time of 30 seconds to 60 minutes, preferably 5 to 45 minutes, particularly preferably 20 to 35 minutes, extremely preferably 25 to 30 minutes.

After the leave-on time for the dye composition (A) has elapsed, the keratin fibers are rinsed with water in order to wash out the dye composition (A).

The keratin fibers can optionally be dried after this rinsing step. The drying can take place using an absorbent cloth, such as a towel. The towel-dried hair can optionally also be dried partially or completely with a hairdryer or other heat source. It is also possible to allow the keratin fibers to dry in the air.

Further preferred dyeing methods according to the invention are characterized in that no oxidation dye precursors are used in them. Typical oxidation dye precursors are p-aminophenol, 4-amino-3-methylphenol, 4-amino-2-aminomethylphenol, 4-amino-2-(1,2-dihydroxyethyl) phenol, 4-amino-2-(diethylaminomethyl) phenol, 2-(2,5-diaminophenyl)ethanol, 2-(1,2-dihydroxyethyl)-p-phenylenediamine, N,N-bis(2-hydroxyethyl)-p-phenylenediamine, N-(4-amino-3-methylphenyl)-N-[3-(1H-imidazol-1-yl)propyl]amine, N,N′-bis(2-hydroxyethyl)-N,N′-bis(4-aminophenyl)-1,3-diaminopropan-2-ol, bis(2-hydroxy-5-aminophenyl) methane, 1,3-bis(2,5-diaminophenoxy)propan-2-ol, N,N′-bis(4-aminophenyl)-1,4-diazacycloheptane, 1,10-bis(2,5-diaminophenyl)-1,4,7,10-tetraoxadecane, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo-[1,2-a]-pyrazol-1-one, 3-aminophenol, 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline and 7-hydroxyindoline.

The dye compositions (A) according to the invention and preferred according to the invention can optionally contain further additives in order to optimize the application properties of these compositions. Preferred additives are in particular thickeners, which ensure that the dye composition (A) stays better on the hair during application.

Dye compositions (A) particularly preferably used according to the invention contain at least one or more hydrophilic thickeners, which are preferably selected from polysaccharides that can be chemically and/or physically modified. Compounds from the group of polysaccharides are particularly preferred according to the invention as hydrophilic thickeners, since the basic structures of the polysaccharides are of natural origin and biodegradable. Preferred hydrophilic polysaccharide thickeners are selected from celluloses, cellulose ethers of C1-C4 alcohols, cellulose esters, xanthan gum, alginic acids (and their corresponding physiologically acceptable salts, the alginates), agar agar (with the polysaccharide agarose present as the main component in agar agar), starch fractions and starch derivatives, such as amylose, amylopectin and dextrins, karaya gum, locust bean gum, gum arabic, pectins, dextrans and guar gum, and mixtures thereof.

Cellulose ethers of C1-C4 alcohols and cellulose esters preferred according to the invention are selected from methyl celluloses, ethyl celluloses, hydroxyalkyl celluloses (such as hydroxyethyl cellulose), methylhydroxyalkyl celluloses, and carboxymethyl celluloses (such as those with the INCI name Cellulose Gum) and also their physiologically acceptable salts.

In preferred embodiments, the hydrophilic thickener present is xanthan gum, with a view to reliable viscosity adjustment and residue-free application to keratin fibers and the scalp. In further preferred embodiments, carboxymethyl cellulose (preferably carboxymethyl cellulose with the INCI name Cellulose Gum) is present as hydrophilic thickener in view of a reliable viscosity adjustment and residue-free application to keratin fibers and the scalp. Carboxymethyl cellulose can be contained in a preferred embodiment as the sole hydrophilic thickener. Particular preference is given to a combination of carboxymethyl cellulose and hydroxyethyl cellulose.

A combination of carboxymethylcellulose and xanthan (preferably xanthan with the INCI name Xanthan Gum) may also be preferred according to the invention.

Dye compositions (A) that are particularly preferred according to the invention contain at least one hydrophilic thickener in a total amount of 0.1 to 5 wt. %, preferably of 0.5 to 4 wt. %, more preferably of 1 to 3.5 wt. %, and very particularly preferably of 1.2 to 2 wt. %, in each case relative to the weight of the respective dye composition (A).

In a further preferred embodiment of the present invention, the dye compositions (A) according to the invention contain, in each case relative to the weight thereof, 0.1 to 3 wt. %, preferably 0.5 to 2.5 wt. %, more preferably 1.0 to 2.0 wt. %, of xanthan gum.

In a further preferred embodiment of the present invention, the dye compositions (A) according to the invention contain, in each case relative to the weight thereof, 0.1 to 4 wt. %, preferably 1 to 2.8 wt. %, of carboxymethyl cellulose.

In a further preferred embodiment of the present invention, the dye compositions (A) according to the invention contain, in each case relative to the weight thereof, 0.1 to 3 wt. %, preferably 0.5 to 2.5 wt. %, more preferably 1.2 to 2.0 wt. %, of hydroxyethyl cellulose.

Dye compositions (A) particularly preferred according to the invention contain at least one organic solvent that has a phenyl group in the molecule. Preferably, this solvent is selected from phenoxyethanol, benzyl alcohol, and mixtures thereof. Surprisingly, it was found that such aromatic solvents can have a positive effect on the dyeing results of the dyeing method according to the invention; this was observed in particular when the dye composition (A) contained such an aromatic solvent.

In a further preferred embodiment of the present invention, the dye compositions (A) preferred according to the invention contain, in each case relative to the weight thereof, 0.1 to 3 wt. %, preferably 0.5 to 2.5 wt. %, more preferably 0.8 to 1.0 wt. %, of at least one organic solvent that has a phenyl group in the molecule. In a further preferred embodiment of the present invention, the dye compositions (A) according to the invention contain, in each case relative to the weight thereof, 0.1 to 3 wt. %, preferably 0.5 to 2.5 wt. %, more preferably 0.8 to 1.0 wt. %, of at least one organic solvent selected from phenoxyethanol, benzyl alcohol and mixtures thereof.

Particularly preferred dye compositions (A) according to the invention are characterized in that they contain at least one aliphatic solvent selected from C₁-C₄ alkanols and C₂-C₄ polyols, in particular selected from ethanol, isopropanol, n-propanol, ethylene glycol, 1,2-propanediol, glycerol and 1,3-butylene glycol, and mixtures of these solvents, but only in a total amount of 0.01-8 wt. %, preferably 0.1-6 wt. %, particularly preferably 0.5-4 wt. %, in each case relative to the weight of the dye composition (A).

Other dye compositions (A) particularly preferred according to the invention are characterized in that they do not contain an aliphatic solvent selected from C₁-C₄ alkanols and C₂-C₄ polyols.

In order to also make the dye compositions (A) used according to the invention sensorially attractive for the user, further dye compositions (A) used particularly preferably according to the invention are characterized in that they contain at least one fragrance oil which contains at least one fragrance compound or odoriferous compound.

The definition of an odorant in the context of the present application corresponds to the definition conventional to one skilled in the art, as can be gathered from RÖMPP Chemie Lexikon [RÖMPP's Chemistry Lexicon], as of December 2007. An odorant, then, is a chemical compound having odor and/or taste, which excites the receptors of the hair cells of the olfactory system (adequate stimulus). The physical and chemical properties necessary for this are a low molar mass of at most 300 g/mol, a high vapor pressure, minimal water solubility and high lipid solubility, as well as weak polarity and the presence of at least one osmophoric group in the molecule. To differentiate volatile, low-molecular-weight substances which are not regarded and used as an odorant, either conventionally and also in the context of the present application, but primarily as a solvent, such as, e.g., ethanol, propanol, isopropanol, and acetone, from odorants according to the invention, odorants according to the invention have a molar mass of 74 to 300 g/mol, contain at least one osmophoric group in the molecule, and have an odor and/or taste; in other words, they stimulate the receptors of the hair cells of the olfactory system. Examples of fragrance and odorant compounds of the ester type are benzyl acetate, phenoxyethyl isobutyrate, ptertbutylcyclohexyl acetate, linalyl acetate, dimethyl benzyl carbinyl acetate (DMBCA), phenyl ethyl acetate, benzyl acetate, ethyl methyl phenyl glycinate, allyl cyclohexyl propionate, styrallyl propionate, benzyl salicylate, cyclohexyl salicylate, Floramat, Melusat, and Jasmecyclat. Dye compositions (A) extremely preferred according to the invention are characterized in that they contain at least one fragrance in a total amount of 0.01-5 wt. %, preferably 0.1-3 wt. %, particularly preferably 0.5-2 wt. %, extremely preferably 1-1.5 wt. %, in each case relative to the weight of the dye composition (A).

Post-treatment steps of the dyeing methods according to the invention

f)i.

In a first embodiment of the dyeing method according to the invention, after rinsing out the dye, the keratin fibers dyed with indigo are rinsed again with additive-free water or are soaked in additive-free water for a period of 5 to 120 minutes, preferably 20 to 110 minutes, particularly preferably 30 to 100 minutes, extremely preferably 45 to 90 minutes.

According to the invention, additive-free water is understood to mean water that does not contain any other added substances. Preferred examples according to the invention are tap water and municipal water. However, deionized water is also considered to be additive-free water.

f)ii.

In a second embodiment of the dyeing method according to the invention, after rinsing out the dye, the keratin fibers dyed with indigo are rinsed again for a period of 5 to 120 minutes with an aqueous cysteine solution (C) having a pH of less than 7.5 measured at 20° C., or are soaked in an aqueous cysteine solution (C) having a pH of less than 7.5 measured at 20° C.

Cysteine, as a chiral amino acid, has a stereogenic center and can occur in a mirror-image form, namely in the form of L-cysteine and D-cysteine. Both L-cysteine and D-cysteine and the mixtures thereof are covered by the present invention. In the context of the present invention, both possible enantiomers can therefore equally be used as a specific compound or else mixtures thereof, and in particular as racemates. However, it is particularly advantageous according to the invention to use the naturally occurring isomer form, in this case L-cysteine.

Cysteine solutions (C) extremely preferably used according to the invention are characterized in that L-cysteine is present in an amount of 0.1-5.0 wt. %, preferably 0.5-3.0 wt. %, particularly preferably 0.8-2.0 wt. %, extremely preferably 1.0-1.5 wt. %, relative to the weight of the cysteine solution (C).

An aqueous solution of cysteine reacts acidically. A 1 wt. % solution of L-cysteine in deionized water has a pH of 6.49, measured at 20° C. Cysteine is therefore not a basifying agent.

Cysteine solutions (C) preferably used have a pH of 2.0 to 7.1, preferably 4.0 to 7.0, particularly preferably 5.0 to 6.6, extremely preferably 5.5 to less than 6.5, in each case measured at 20° C.

f)iii.

In a third embodiment of the dyeing method according to the invention, after rinsing out the dye, the keratin fibers dyed with indigo are rinsed again for a period of 5 to 120 minutes with an aqueous composition (G) containing at least one glycosylase (EC 3.2) or are soaked in an aqueous composition (G) containing at least one glycosylase (EC 3.2).

### Glycosylases (EC 3.2)

In a preferred embodiment of the invention, the at least one glycosylase (EC 3.2) is selected from at least one glycosidase (EC 3.2.1). Glycosidases (EC 3.2.1) are understood to mean enzymes that hydrolyze O-glycosyl components and S-glycosyl components.

Glycosidases (EC 3.2.1) preferred according to the invention are selected from a group of enzymes generally referred to as cellulases.

The term “cellulase” as used herein refers to enzymes that catalyze the hydrolysis of 1,4-beta-D-glucoside bonds that are present in cellulose (cellobiose) and/or lichenin and/or beta-D-glucans. Cellulases are often also able to hydrolyze the 1,4 bonds in beta-D-glucans, which also have 1,3 bonds in addition to the 1,4 bonds. Cellulases are able to break down cellulose to beta-glucose. Consequently, cellulases act in particular upon cellulose-containing or cellulose derivative-containing functional groups and catalyze their hydrolysis. The determining factor as to whether an enzyme is a cellulase in the context of the invention is its ability to hydrolyze 1,4-beta-D-glucoside bonds in cellulose.

The term “cellulase activity” is defined here as an enzyme that catalyzes the hydrolysis of 1,4-beta-D-glucoside bonds in beta-1,4-glucan (cellulose). Cellulose activity is measured using a standard method, e.g., as follows: Cellulases release glucose from CMC (carboxymethylcellulose). The samples are incubated under defined reaction conditions (100 mM sodium phosphate buffer pH 7.5, 40° C., 15 min) with a substrate (1.25 wt. % CMC). The reaction with p-hydroxybenzoic acid hydrazide (PAHBAH) in the presence of bismuth produces a yellow dye that can be determined photometrically at 410 nm. The prerequisite is an alkaline pH during the color reaction. The amount of sugar released corresponding to the coloration is a measure of enzyme activity (Lever, Anal. Biochem., 1972, 47 & 1977, 81).

Cellulases can be divided into three categories:
1. Endoglucanase (EC 3.2.1.4), also called endo-1,4-beta-glucanase, beta-1,4-glucanase, avicelase, beta-1,4-endoglucanhydrolase, endo-1,4-beta-D-glucanohydrolase, carboxymethylcellulase, or celludextrinase;
2. Cellulose-1,4-beta-cellobiosidase (non-reducing end) (EC 3.2.1.91), also called exoglucanase, 1,4-beta-cellobiohydrolase, 4-beta-D-glucan cellobiohydrolase (non-reducing end), avicelase, exo-1,4-beta-D-glucanase, or exocellobiohydrolase, releases cellobiose from the non-reducing ends of the B-D-glucan chains by hydrolysis of the (1->4)-beta-D-glucosidic bonds in cellulose and cellotetraose;
3. beta-glucosidase (EC 3.2.1.21), also called cellobiase, beta-D-glucoside glucohydrolase, amygdalase, or gentobiase, hydrolyzes terminal, non-reducing beta-D-glucosyl groups to release beta-D-glucose.

In a further preferred embodiment of the invention, the at least one glycosidase (EC 3.2.1) is selected from endo-1,4-beta-glucanase (EC 3.2.1.4), beta-glucosidase (EC 3.2.1.21), exo-1,4-beta-glucosidase (EC 3.2.1.74), cellulose-1,4-beta-cellobiosidase (EC 3.2.1.176), exo-1,4-beta-D-glucanase (EC 3.2.1.91), and oligoxyloglucan reducing-end-specific cellobiohydrolase (EC 3.2.1.150), and mixtures of these enzymes.

In a further preferred embodiment of the invention, the at least one glycosidase (EC 3.2.1) is selected from endo-1,4-beta-glucanase (EC 3.2.1.4), beta-glucosidase (EC 3.2.1.21), and exo-1,4-beta-D-glucanase (EC 3.2.1.91), and mixtures of these enzymes.

A particularly preferred embodiment of the invention is characterized in that the composition (G) used according to the invention contains an enzyme mixture comprising at least one endo-1,4-beta-glucanase (EC 3.2.1.4), at least one beta-glucosidase (EC 3.2.1.21), and at least one enzyme selected from at least one exo-1,4-beta-D-glucanase (EC 3.2.1.91) and at least one oligoxyloglucan reducing-end-specific cellobiohydrolase (EC 3.2.1.150), and mixtures thereof.

A particularly preferred embodiment of the invention is characterized in that the composition (G) contains an enzyme mixture comprising at least one endo-1,4-beta-glucanase (EC 3.2.1.4), at least one beta-glucosidase (EC 3.2.1.21), and at least one exo-1,4-beta-D-glucanase (EC 3.2.1.91).

A further particularly preferred embodiment of the invention is characterized in that the composition (G) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and—in a total amount of 25-35 wt. %—at least one enzyme selected from at least one exo-1,4-beta-D-glucanase (EC 3.2.1.91) and at least one oligoxyloglucan reducing-end-specific cellobiohydrolase (EC 3.2.1.150), and mixtures thereof, wherein the indicated amounts are relative to the total weight of the enzyme mixture.

A further particularly preferred embodiment of the invention is characterized in that the composition (G) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and at least one exo-1,4-beta-D-glucanase (EC 3.2.1.91) in a total amount of 25-35 wt. %, wherein the indicated amounts are relative to the total weight of the enzyme mixture.

A further particularly preferred embodiment of the invention is characterized in that the composition (G) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, furthermore—in a total amount of 25-35 wt. %—at least one enzyme selected from at least one exo-1,4-beta-D-glucanase (EC 3.2.1.91) and at least one oligoxyloglucan reducing-end-specific cellobiohydrolase (EC 3.2.1.150), and mixtures thereof, as well as at least one other enzyme with cellulase activity in a total amount up to 100 wt. %, wherein the indicated amounts are relative to the total weight of the enzyme mixture.

A further particularly preferred embodiment of the invention is characterized in that the composition (G) contains an enzyme mixture comprising at least one endo-1,4-beta-glucanase (EC 3.2.1.4) in a total amount of 20-35 wt. %, at least one beta-glucosidase (EC 3.2.1.21) in a total amount of 15-25 wt. %, and at least one exo-1,4-beta-D-glucanase (EC 3.2.1.91) in a total amount of 25-35 wt. %, as well as at least one other enzyme with cellulase activity in a total amount up to 100 wt. %, wherein the indicated amounts are relative to the total weight of the enzyme mixture.

Compositions (G) preferably used according to the invention contain, in each case relative to the weight thereof, at least one glycosylase (EC 3.2) in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

A further particularly preferred embodiment of the invention is characterized in that the composition (G) contains, relative to the weight thereof, at least one glycosidase (EC 3.2.1), selected from at least one cellulase, in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

A further particularly preferred embodiment of the invention is characterized in that the composition (G) contains, relative to the weight thereof, at least one glycosidase (EC 3.2.1), selected from endo-1,4-beta-glucanase (EC 3.2.1.4), beta-glucosidase (EC 3.2.1.21), and exo-1,4-beta-D-glucanase (EC 3.2.1.91) and mixtures of these enzymes, in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

A further particularly preferred embodiment of the invention is characterized in that the composition (G) contains, relative to the weight thereof, at least one glycosidase (EC 3.2.1), selected from at least one endo-1,4-beta-glucanase (EC 3.2.1.4), at least one beta-glucosidase (EC 3.2.1.21), and at least one enzyme selected from at least one exo-1,4-beta-D-glucanase (EC 3.2.1.91) and at least one oligoxyloglucan reducing-end-specific cellobiohydrolase (EC 3.2.1.150) and also mixtures thereof, in a total amount of 0.0001-1 wt. %, preferably 0.001-0.1 wt. %, more preferably 0.002-0.05 wt. %, particularly preferably 0.003-0.04 wt. %, extremely preferably 0.01-0.03 wt. %.

In compositions (G) preferably used according to the invention, the water content is 40-99 wt. %, preferably 50-97 wt. %, particularly preferably 60-90 wt. %, in each case relative to the weight of the composition (G).

f)iv.

In a fourth embodiment of the dyeing method according to the invention, after rinsing out the dye, the keratin fibers dyed with indigo are rinsed again for a period of 5 to 120 minutes with an aqueous composition (L) containing laccase (EC 1.10.3.2) or are soaked in an aqueous composition (L) containing laccase (EC 1.10.3.2).

Compositions (L) preferably used according to the invention contain, in each case relative to the weight thereof, 0.0001-1 wt. %, preferably 0.001b-0.3 wt. %, more preferably 0.05-0.2 wt. %, particularly preferably 0.06-0.15 wt. %, extremely preferably 0.07-0.1 wt. % laccase (EC 1.10.3.2).

In compositions (L) preferably used according to the invention, the water content is 40-99 wt. %, preferably 50-97 wt. %, particularly preferably 60-90 wt. %, in each case relative to the weight of the composition (L).

f)v.

In a fourth embodiment of the dyeing method according to the invention, after rinsing out the dye, the keratin fibers dyed with indigo are rinsed again for a period of 5 to 120 minutes with an aqueous composition (AT) which, relative to the weight thereof, contains 1-20 wt. % of an anionic surfactant and has a pH in the range of 3-6, measured at 20° C., or are soaked in an aqueous composition (AT) which, relative to the weight thereof, contains 1-20 wt. % of an anionic surfactant and has a pH in the range of 3-6, measured at 20° C.

Suitable anionic surfactants for the agents according to the invention are all anionic surface-active substances suitable for use on the human body which have a water-solubilizing anionic group, for example a carboxylate, sulfate, sulfonate or phosphate group, and a lipophilic alkyl group having approximately 8 to 30 C atoms, preferably 8 to 24 C atoms in the molecule, with the exception of linear and branched fatty acids having 8 to 30 C atoms and the salts thereof (soaps). In addition, glycol ether or polyglycol ether groups, ester, ether and amide groups, and hydroxyl groups can be contained in the molecule. Examples of suitable anionic surfactants are, in each case in the form of the sodium, potassium and ammonium salts and also mono-, di- and trialkanolammonium salts having 2 to 4 C atoms in the alkanol group, polyethoxylated ether carboxylic acids, acyl sarcosides, acyl taurides, acyl isethionates, sulfosuccinic acid mono- and dialkyl esters and sulfosuccinic acid monoalkyl polyoxyethyl esters having 1 to 6 ethylene oxide groups, linear alkanesulfonates, linear alpha-olefinsulfonates, sulfonates of unsaturated fatty acids having up to 6 double bonds, alpha-sulfofatty acid methyl esters of fatty acids, C₈-C₂₀ alkyl sulfates and C₈-C₂₀ alkyl ether sulfates having 1 to 15 oxyethyl groups, mixtures of surface-active hydroxysulfonates, sulfated hydroxyalkyl polyethylene and/or hydroxyalkylene propylene glycol ethers, esters of tartaric acid or citric acid with ethoxylated or propoxylated fatty alcohols, optionally polyethoxylated alkyl and/or alkenyl ether phosphates, sulfated fatty acid alkylene glycol esters, and monoglyceride sulfates and monoglyceride ether sulfates. Preferred anionic surfactants are selected from C₈-C₂₀ alkyl sulfates, C₈-C₂₀ alkyl ether sulfates and C₈-C₂₀ ether carboxylic acids, each having 8 to 20 C atoms in the alkyl group and 0 to 12 ethylene oxide groups in the molecule. Sodium laureth (2) sulfate is particularly preferred.

The anionic surfactant composition (AT) used according to the invention has a pH in the range of 3-6, preferably 4-5.5, particularly preferably 4.5-5, in each case measured at 20° C.

### EMBODIMENTS

The exemplary embodiments presented below are intended to explain the subject matter of the invention in more detail, without limiting it thereto.

The color difference, also known as dE or ΔE, can be easily determined colorimetrically using a colorimeter that measures colors in the L*, a*, b* color space—for example, a Datacolor colorimeter, type Spectraflash SF 600. All colorimetric measurements described below were carried out using the Spectraflash SF 600 colorimetric device from Datacolor.

The L*, a, b* color space refers to the CIELAB color space. The L value represents the brightness of the color (black-white axis); the greater the L value, the brighter the color. The a value represents the red-green axis of the system; the greater this value, the more the color is shifted towards red. The b value represents the yellow-blue axis of the system; the greater this value, the more the color is shifted towards yellow.

The color shift ΔE, i.e., the color difference between two (hair) colors, for each of which an L*, a*, b* value combination was determined, is calculated according to the following formula:

$$Δ E = {( Δ {L}^{2} + Δ {a}^{2} + Δ {b}^{2} )}^{0.5}$$

The greater the value for ΔE, the more pronounced the color difference.

For the spectrophotometer measurements, a D65 illuminant and a diffuse/8° optical configuration were used. The spectral reflectance data for each sample from 380 nm to 700 nm were converted to colorimetric data using DCI Color software. Reflectance measurements were determined for each hair sample, with the average of 4 measurements being recorded.

The color difference (ΔE) between untreated strand and colored treated was calculated according to the following formula:

$$ΔE = \sqrt{{( L v - L n )}^{2} + {( a v - a n )}^{2} + {( b v - b n )}^{2}} ,$$

where
Lv, av, bv: colorimetric values for untreated strand
Ln, an, bn: colorimetric values for treated strand

In order to track the development of the final color over time, measurements were taken on day 0 immediately after dyeing and 14 days after dyeing and compared with each other.

To prepare the dye composition (A) used according to the invention, 2.5 g of indigo powder were dispersed in 47.5 grams of demineralized water (5 wt. % of the total dispersion). After stirring the dispersion for 2 minutes, a strand of buffalo belly hair (brush body with round binding, approx. 8 cm of free hair) was placed in the dye composition (A) for 30 minutes and stirred (liquor ratio: 50 grams of dye composition to 1 gram of hair strand, pH: 6.46-7.29). Subsequently, the dye composition (A) was washed off the strands by rinsing the strands under running deionized water (20° C.) for 30 seconds while combing 20 times. The hair strands were then dried with a commercially available hair dryer at a defined distance (d=10 cm) and a defined temperature (T=80±5° C.) while combing them 20 times.

The control strands were also treated as described above and were not further post-treated.

The following plant raw materials were used:
“C30 Indigo Leaves Powder” (Indigofera tinctoria L.): powdered leaves of Indigofera tinctoria, pH of the 5 wt. % dispersion in water: 6.8 to 7.2 (20° C.)
   Source: Couleurs de Plantes, rue de l'Arsenal, 17300 Rochefort, France or
   “Indigo Powder” (Indigofera tinctoria L.): powdered leaves of Indigofera tinctoria, pH of the 5 wt. % dispersion in water: 6.8 to 7.2 (20° C.)
   Source: Matha Exports International LLP, Ashram, India

Immediately thereafter, i.e. within a time of zero seconds to 30 minutes, preferably 10 seconds to 10 minutes, particularly preferably 1 to 5 minutes, after drying the strands, one of five different post-treatments was carried out—except for on the control strands—as described below.

f)i. Post-Treatment with Water

A first embodiment of the dyeing method according to the invention comprises the post-treatment of the freshly indigo-dyed strands with pure, i.e. additive-free, demineralized water. To this end, the freshly dyed indigo strands were placed in 50 ml of demineralized water (20° C., liquor ratio: 50 grams of additive-free demineralized water to 1 gram of hair strand). These were stirred therein for 1.5 hours and then combed and dried as described above.

Immediately after the post-treatment, a slightly less green and slightly bluer color tone (ΔE 6.1) could be observed compared to the control strand.

The ΔE value between the mean values of the freshly dyed strands with water post-treatment and the strands 14 days after dyeing and water post-treatment is 1.8. The corresponding ΔE value of the control strands between both measurement times is 18.5. The differences between the control strand and the strands with water post-treatment are listed below (individual measurements, mean values and ΔE values: see Table 1).

TABLE 1
L*, a*, b*, ΔE values (mean values) of indigo dyeings
with water post-treatment (according to the invention)
and without water post-treatment (control)

| | | | |
|---|---|---|---|
| | CIE L | CIE a | CIE b |
| Batch Name | specimen | specimen | specimen |

Day 0 (immediately after dyeing)

| | | | |
|---|---|---|---|
| Indigo dyeing without water post- | 45.4 | −13.0 | −6.8 |
| treatment (control) | | | |
| Indigo dyeing with water post- | 43.1 | −7.4 | −7.4 |
| treatment (according to the | | | |
| invention) | | | |

Difference between control and water strand: ΔE = 6.1
Day 14

| | | | |
|---|---|---|---|
| Indigo dyeing without water post- | 36.0 | 1.4 | −13.6 |
| treatment (control) | | | |
| Indigo dyeing with water post- | 42.5 | −6.2 | −8.6 |
| treatment (according to the | | | |
| invention) | | | |

Difference between control strands and water strands: ΔE = 11.2
Difference between water strands day 0 and day 14: ΔE = 1.8
Difference between control strands day 0 and day 14: ΔE = 18.5

Even after 14 days, the strands post-treated with water are colored blue and exhibit a significantly less pronounced red/purple tone than the control strands (see Table 1). Due to the post-treatment with water, the formation of the red tint is significantly reduced or stopped after 14 days.

The water post-treatment achieves a clearer shade of blue right from the start. After dyeing the strand, the color result remains more constant and more stable, with a ΔE of 1.8, than the control strand with a ΔE of 18.5.

f)ii. Post-Treatment with Cysteine

A second embodiment of the dyeing method according to the invention comprises the post-treatment of the freshly indigo-dyed strands with an aqueous cysteine solution (C).

To this end, 0.5 g of (R)-(+)-cysteine was dissolved in 49.5 grams of demineralized water (1 wt. % of the total solution, pH: 5.47), stirred for 2 minutes and the freshly dyed indigo strands were added (liquor ratio: 50 grams of cysteine solution (C) to 1 g of hair strand).

These were stirred therein for 30 minutes and then combed and dried as described above.

Directly after the post-treatment, a slightly less green and slightly bluer color tone (ΔE 6.3) could be observed compared to the control strand.

The ΔE value between the mean values of the freshly dyed strands with cysteine post-treatment and the strands 14 days after dyeing and cysteine post-treatment is 3.9. The corresponding ΔE value of the control between both measurement times is 18.5. The differences between the control strand and the strands with cysteine post-treatment are listed below (individual measurements, mean values and ΔE values: see Table 2).

TABLE 2
L*, a*, b*, ΔE values (mean values) of indigo dyeings
with cysteine post-treatment (according to the invention)
and without cysteine post-treatment (control)

| | | | |
|---|---|---|---|
| | CIE L | CIE a | CIE b |
| Batch Name | specimen | specimen | specimen |

Day 0 (immediately after dyeing)

| | | | |
|---|---|---|---|
| Indigo dyeing without cysteine | 45.4 | −13.0 | −6.8 |
| post-treatment (control) | | | |
| Indigo dyeing with cysteine post- | 47.8 | −7.7 | −9.3 |
| treatment (according to the | | | |
| invention) | | | |

Difference between control and cysteine strand: ΔE = 6.3
Day 14

| | | | |
|---|---|---|---|
| Indigo dyeing without cysteine | 36.0 | 1.4 | −13.6 |
| post-treatment (control) | | | |
| Indigo dyeing with cysteine post- | 46.6 | −4.1 | −10.0 |
| treatment (according to the | | | |
| invention) | | | |

Difference between control strands and cysteine strands: ΔE = 12.5
Difference between cysteine strands day 0 and day 14: ΔE = 3.9
Difference between control strands day 0 and day 14: ΔE = 18.5

Even after 14 days, the strand post-treated with cysteine is colored blue and exhibits a significantly less pronounced red/purple tone than the control strands (see Table 2). Due to the post-treatment with cysteine, the formation of the red tint is significantly reduced or stopped after 14 days.

The post-treatment with 1 wt. % cysteine in aqueous solution achieves a clearer blue tone right from the start. After dyeing the strand, the color result remains more stable, with a ΔE of 3.9, than the control strand with a ΔE of 18.5.

f)iii. Post-Treatment with Glycosylase

A third embodiment of the dyeing method according to the invention comprises the post-treatment of the freshly indigo-dyed strands with an aqueous composition (G) containing at least one glycosylase (EC 3.2).

To produce the composition (G), a glycosylase mixture (EC 3.2) was used, obtained from Novozymes, hereinafter referred to as “cellulase blend”. This enzyme mixture comprised at least one endo-1,4-beta-glucanase (EC 3.2.1.4) in a total amount of 20-35 wt. %, at least one beta-glucosidase (EC 3.2.1.21) in a total amount of 15-25 wt. %, furthermore at least one enzyme selected from at least one exo-1,4-beta-D-glucanase (EC 3.2.1.91) and at least one oligoxyloglucan reducing-end-specific cellobiohydrolase (EC 3.2.1.150) and mixtures thereof in a total amount of 25-35 wt. %, as well as at least one other enzyme with glycosylase activity in a total amount up to 100 wt. %, wherein the indicated amounts are relative to the total weight of the enzyme mixture. The enzyme active substance content in the raw material used, which was present as an aqueous composition, was 190 μg/μl, i.e. 19 wt. %. Furthermore, the “cellulase blend” contained, relative to the weight thereof, 66 wt. % water and 15 wt. % of a sucrose/D-glucose mixture.

To prepare composition (G), 100 μl of “cellulase blend” were made up to a 50 gram solution (0.038 wt. % enzyme content, pH 7 (20° C.)) with demineralized water, stirred for 2 minutes and the freshly dyed indigo strands were added (liquor ratio: 50 grams of solution to 1 gram of hair strand). These were stirred therein for 30 minutes and then combed and dried as described above.

Immediately after the post-treatment, a slightly less green color tone (ΔE 4.1) could be observed compared to the control strand.

The ΔE value between the mean values of the freshly dyed cellulase post-treated strands and the cellulase post-treated strands 14 days after dyeing and cellulase post-treatment is 4.8. The corresponding ΔE value of the control between both measurement times is 18.5. The differences between the control strand and the strands with the addition of cellulase are listed below (individual measurements, mean values and ΔE values: see Table 3).

TABLE 3
L*, a*, b*, ΔE values (mean values) of indigo dyeings
with cellulase post-treatment (according to the invention)
and without cellulase post-treatment (control)

| | | | |
|---|---|---|---|
| | CIE L | CIE a | CIE b |
| Batch Name | specimen | specimen | specimen |

Day 0 (immediately after dyeing)

| | | | |
|---|---|---|---|
| Indigo dyeing without cellulase | 45.4 | −13.0 | −6.8 |
| post-treatment (control) | | | |
| Indigo dyeing with cellulase post- | 45.0 | −8.9 | −6.9 |
| treatment (according to the | | | |
| invention) | | | |

Difference between control and cellulase strand: ΔE = 4.1
Day 14

| | | | |
|---|---|---|---|
| Indigo dyeing without cellulase | 36.0 | 1.4 | −13.6 |
| post-treatment (control) | | | |
| Indigo dyeing with cellulase post- | 43.3 | −5.0 | −9.1 |
| treatment (according to the | | | |
| invention) | | | |

Difference between control strands and cellulase strands: ΔE = 10.7
Difference between cellulase strands day 0 and day 14: ΔE = 4.8
Difference between control strands day 0 and day 14: ΔE = 18.5

Even after 14 days, the strand post-treated with cellulase is colored blue and exhibits a significantly less pronounced red/purple tone than the control strands (see Table 3). Due to the post-treatment with cellulase, the formation of the red tint is significantly reduced or stopped after 14 days.

The cellulase post-treatment achieves a clearer shade of blue right from the start. After dyeing the strand, the color result remains more stable, with a ΔE of 4.8, than the control strand with a ΔE of 18.5.

f)iv. Post-Treatment with Laccase

A fourth embodiment of the dyeing method according to the invention comprises the post-treatment of the freshly indigo-dyed strands with an aqueous composition (G) containing laccase (EC 1.10.3.2).

To prepare the aqueous composition (L), an aqueous laccase preparation (EC 1.10.3.2) from Novozymes was used, which had an active substance content of 6.25 wt. %.

To prepare the aqueous composition (L), 600 μl of laccase preparation were made up to 50 grams (0.075 wt. % enzyme content, pH 7 (20° C.)) with demineralized water, stirred for 2 minutes and the freshly dyed indigo strands were added (liquor ratio: 50 grams of laccase solution (L) to 1 gram of hair strand). These were stirred therein for 30 minutes and then combed and dried as described above.

Immediately after the post-treatment, a slightly less green and slightly bluer color tone (ΔE 4.6) could be observed compared to the control strand. The ΔE value between the mean values of the freshly dyed strands post-treated with laccase and the strands post-treated with laccase 14 days after dyeing and laccase post-treatment is 7.0. The corresponding ΔE value of the control between both measurement times is 18.5. The differences between the control strand and the strands with the addition of laccase are listed below (individual measurements, mean values and ΔE values: see Table 4).

TABLE 4
L*, a*, b*, ΔE values (mean values) of indigo dyeings
with laccase post-treatment (according to the invention)
and without laccase post-treatment (control)

| | | | |
|---|---|---|---|
| | CIE L | CIE a | CIE b |
| Batch Name | specimen | specimen | specimen |

Day 0 (immediately after dyeing)

| | | | |
|---|---|---|---|
| Indigo dyeing without laccase post- | 45.4 | −13.0 | −6.8 |
| treatment (control) | | | |
| Indigo dyeing with laccase post- | 45.7 | −8.4 | −7.1 |
| treatment (according to the invention) | | | |

Difference between control and laccase strand: ΔE = 4.6
Day 14

| | | | |
|---|---|---|---|
| Indigo dyeing without laccase post- | 36.0 | 1.4 | −13.6 |
| treatment (control) | | | |
| Indigo dyeing with laccase post- | 42.7 | −2.8 | −10.0 |
| treatment (according to the invention) | | | |

Difference between control strands and laccase strands: ΔE = 8.7
Difference between laccase strands day 0 and day 14: ΔE = 7.0
Difference between control strands day 0 and day 14: ΔE = 18.5

After 14 days, the strand post-treated with laccase is colored blue and exhibits a less pronounced red/purple tone than the control strands (see Table 4). Due to the post-treatment with laccase, the formation of the red tint is reduced after 14 days.

The laccase post-treatment achieves a clearer shade of blue right from the start. After dyeing the strand, the color result remains more stable, with a ΔE of 7.0, than the control strand with a ΔE of 18.5.

f)v. Post-Treatment with Acidic Anionic Surfactant Solution

A fifth embodiment of the dyeing method according to the invention comprises the post-treatment of the freshly indigo-dyed strands with an aqueous composition (AT) which, relative to the weight thereof, contains 1-20 wt. % of an anionic surfactant and has a pH in the range of 3-6, measured at 20° C.

To produce the aqueous composition (AT), an aqueous solution of 12.5 wt. % sodium laureth sulfate was produced and adjusted to a pH of 4.5 (20° C.) with citric acid, and the freshly dyed indigo strands were added (liquor ratio: 50 grams of anionic surfactant solution (AT) to 1 g of hair strand). These were stirred therein for 30 minutes and then combed and dried as described above.

Immediately after the anionic surfactant post-treatment, a slightly less green and slightly bluer color tone (ΔE 6.8) could be observed compared to the control strand.

The ΔE value between the mean values of the freshly dyed strands post-treated with the anionic surfactant and these same strands 14 days after dyeing and post-treatment is 6.1. The corresponding ΔE value of the control between both measurement times is 18.5. The differences between the control strand and the strands with test wash post-treatment are listed below (individual measurements, mean values and ΔE values: see Table 5).

TABLE 5
L*, a*, b*, ΔE values (mean values) of indigo dyeings with
anionic surfactant post-treatment (according to the invention)
and without anionic surfactant post-treatment (control)

| | | | |
|---|---|---|---|
| | CIE L | CIE a | CIE b |
| Batch Name | specimen | specimen | specimen |

Day 0 (immediately after dyeing)

| | | | |
|---|---|---|---|
| Indigo dyeing without anionic | 45.4 | −13.0 | −6.8 |
| surfactant post-treatment (control) | | | |
| Indigo dyeing with anionic | 43.9 | −6.9 | −9.5 |
| surfactant post-treatment | | | |
| (according to the invention) | | | |

Difference between control and anionic surfactant strand: ΔE = 6.8
Day 14

| | | | |
|---|---|---|---|
| Indigo dyeing without anionic | 36.0 | 1.4 | −13.6 |
| surfactant post-treatment (control) | | | |
| Indigo dyeing with anionic | 41.0 | −1.4 | −11.6 |
| surfactant post-treatment | | | |
| (according to the invention) | | | |

Difference between control strands and anionic surfactant strands: ΔE =
6.1
Difference between anionic surfactant strands day 0 and day 14: ΔE = 6.6
Difference between control strands day 0 and day 14: ΔE = 18.5

After 14 days, the strand post-treated with anionic surfactant is colored blue and exhibits a less pronounced red/purple tone than the control strands (see Table. 5s). The anionic surfactant post-treatment reduces the formation of the red tint after 14 days.

The anionic surfactant post-treatment achieves a clearer blue tone right from the start. After dyeing the strand, the color result remains more stable, with a ΔE of 6.6, than the control strand with a ΔE of 18.5.

1. A method for non-oxidative dyeing of keratin fibers, the method comprising:
providing an aqueous dye composition comprising at least one indigo-producing plant powder, wherein the aqueous dye composition is free of cysteine and has a pH of from 2.0 to 10.0 measured at 20° C.;
applying the aqueous dye composition to the keratin fibers;
maintaining the aqueous dye composition in the keratin fibers for a time of from 30 seconds to 60 minutes;
rinsing the keratin fibers; and
treating the keratin fibers by rinsing or soaking the keratin fibers for a time of from 5 minutes to 120 minutes with at least one of:
additive-free water,
an aqueous cysteine solution having a pH of less than 7.5 measured at 20° C.,
an aqueous composition comprising at least one glycosylase,
an aqueous composition comprising laccase, or
an aqueous composition comprising an anionic surfactant in an amount of from 1-20 wt. %, based on the total weight of the aqueous composition comprising the anionic surfactant, and having a pH of from 3-6 measured at 20° C.,
wherein no hydrogen peroxide and no ions and compounds of metals other than alkali metals and alkaline-earth metals are used in the method.

2. The method of claim 1, wherein maintaining the aqueous dye composition in the keratin fibers is in the presence of heat at a temperature of from 25-60° C.

3. The method of claim 1, wherein the at least one indigo-producing plant powder is powder from at least one indigo-producing plant selected from at least one species of the group consisting of:
Indigofera,
Isatis,
Persicaria,
Wrightia,
Calanthe, and
Baphicacanthus cusia.

4. The method of claim 1, wherein the at least one indigo-producing plant powder comprises leaves of at least one indigo-producing plant in an amount of at least 50 wt. %, based on the total weight of the at least one indigo-producing plant powder.

5. The method of claim 1, wherein the at least one indigo-producing plant powder comprises indican in an amount of from 2-5 wt. %, based on the total weight of the at least one indigo-producing plant powder.

6. The method of claim 1, wherein the at least one indigo-producing plant powder is in an amount of from 1-80 wt. %, based on the total weight of the aqueous dye composition.

7. The method of claim 6, wherein the at least one indigo-producing plant powder comprises powdered leaves of the plant species Indigofera tinctoria.

8. The method of claim 1, wherein the aqueous dye composition further comprises water in an amount of from 19.9-95.0 wt. %, based on the total weight of the aqueous dye composition.

9. The method of claim 1, wherein the aqueous cysteine solution comprises cysteine in an amount of from 0.1-5 wt. %, based on the total weight of the aqueous cysteine solution.

10. The method of claim 1, wherein the aqueous cysteine solution has a pH of from 2.0 to 7.1 measured at 20° C.

11. The method of claim 1, wherein the at least one glycosylase is selected from is at least one glycosidase.

12. The method of claim 11, wherein the at least one glycosidase is at least one cellulase.

13. The method of claim 11, wherein the at least one glycosidase is selected from the group consisting of endo-1,4-beta-glucanase, beta-glucosidase, exo-1,4-beta-glucosidase, cellulose-1,4-beta-cellobiosidase, exo-1,4-beta-D-glucanase, oligoxyloglucan reducing-end-specific cellobiohydrolase, and any mixture thereof.

14. The method of claim 1, wherein the composition comprising at least one glycosylase comprises the at least one glycosylase in an amount of from 0.0001-1 wt. %, based on the total weight of the composition comprising at least one glycosylase.

15. The method of claim 1, wherein the composition comprising laccase comprises the laccase in an amount of from 0.0001-1 wt. %, based on the total weight of the composition comprising laccase.

16. The method of claim 1, wherein the keratin fibers are human hair.

17. The method of claim 1, further comprising drying the keratin fibers before treating the keratin fibers.

18. The method of claim 1, wherein the aqueous dye composition has a pH of from 6.0 to 7.4 measured at 20° C.

19. The method of claim 1, wherein the at least one indigo-producing plant powder is in an amount of from 6-7 wt. %, based on the total weight of the aqueous dye composition.

20. The method of claim 3, wherein the at least one indigo-producing plant powder is a powder of the plant species Indigofera tinctoria.
